(19) United States
(12) Patent Application Publication
(10) Pub. No.: US 2023/0242910 A1
(43) Pub. Date: Aug. 3, 2023

(54) METHODS AND COMPOSITIONS RELATING TO ENGINEERED GUIDE SYSTEMS FOR ADENOSINE DEAMINASE ACTING ON RNA EDITING

(71) Applicant: SHAPE THERAPEUTICS INC., Seattle, WA (US)

(72) Inventors: Yiannis Savva, Seattle, WA (US); Richard Thomas Sullivan, Seattle, WA (US); Stephen Michael Burleigh, Seattle, WA (US)

(21) Appl. No.: 17/927,385

(22) PCT Filed: May 25, 2021
(86) PCT No.: PCT/US2021/034099

§ 371 Date: Nov. 23, 2022

Related U.S. Application Data
(60) Provisional application No. 63/030,030, filed on May 26, 2020.

(51) Int. Cl. C12N 15/113; C12N 9/78; C12N 15/85

(52) U.S. Cl. CPC C12N 15/113; C12N 9/78; C12N 15/85; C12Y 305/04004; C12N 2310/15; C12N 2310/531; C12N 2320/33; C12N 2800/107

(57) ABSTRACT

Provided herein are compositions of engineered guide RNAs relating to providing or engineering a structural target to attract adenosine deaminase acting on RNA (ADAR) editing to a desired site and methods of use thereof. Further provided herein are compositions and methods relating to recombinant adenosine deaminase acting on RNA (ADAR) split guide RNAs (adar-sgRNA). In certain embodiments, the adar-sgRNA composition comprises two guides-one guide with an ADAR recruiting domain and a second guide with a 5′ and/or 3′ RNA targeting domain for forming a trimolecular complex at a mismatch site that serves to recruit ADAR. Binding of the two adar-split gRNAs to the target RNA forms a trimolecular complex which recruits ADAR enzymes to deaminate one or more mismatched adenosine residues in the adar-split gRNA RNA targeting domain: target RNA duplex. In certain embodiments, such compositions and methods will be useful for modifying a coding sequence of a desired protein.

### 1. CROSS-REFERENCE TO RELATED APPLICATIONS

This application claims the benefit of and priority to U.S. Provisional Pat. Application No. 63/030,030 filed on May 26, 2020, which is incorporated by reference in its entirety.

### 2. SEQUENCE LISTING

The instant application contains a Sequence Listing with XX sequences, which has been submitted via EFS-Web and is hereby incorporated herein by reference in its entirety. Said ASCII copy, created on XXXX, is named 46343WO_sequencelisting.txt, and is XXX bytes in size.

### 3. BACKGROUND

RNA editing is a post-transcriptional process that recodes hereditary information by changing the nucleotide sequence of RNA molecules. One of the most prevalent forms of post-transcriptional RNA modification is the conversion of adenosine-to-inosine (A-to-I), mediated by adenosine deaminase acting on RNA (ADAR) enzymes. Adenosine-to-inosine (A-to-I) RNA editing alters genetic information at the transcript level and is a biological process commonly conserved in metazoans. A-to-I editing is catalyzed by adenosine deaminase acting on RNA (ADAR) enzymes. Such intracellular RNA-editing mechanisms can potentially provide a versatile RNA-mutagenesis method for genome manipulation.

Current systems used to edit RNA rely on the delivery of non-encodable components and have limitations which may lead to aberrant effector activity, delivery barrier, unintended transcriptomic modifications, or immunogenicity. Thus, further methods and systems for improved efficiency, specificity, and safety of targeted RNA editing are needed.

### 4. SUMMARY

In an aspect, the present disclosure provides a recombinant adenosine deaminase acting on RNA (ADAR) split guide RNA (adar-sgRNA), comprising a first guide RNA comprising a 5′ segment of the ADAR recruiting domain, a 5′ RNA targeting domain capable of binding to a target RNA, or both domains, a second guide RNA comprising a 3′ segment of the ADAR recruiting domain, a 3′ RNA targeting domain capable of binding to a target RNA, or both domains, wherein at least one RNA targeting domain has a sequence that is at least partially complementary to the sequence of a segment of the target RNA, wherein the binding of the first guide RNA and the second guide RNA to the target RNA splints the first and second guide RNAs with the target RNA forming a trimolecular complex, and wherein the binding of the first guide RNA and the second guide RNA to the target RNA splints the first and second guide RNAs with the target RNA forming a trimolecular complex, and wherein the trimolecular complex is capable of recruiting ADAR to deaminate one or more adenosines in the target RNA.

In certain embodiments, the formation of the trimolecular complex reconstitutes an ADAR recruiting domain comprising the 5′ segment of the ADAR recruiting domain and the 3′ segment of the ADAR recruiting domain.

In certain embodiments, the first guide RNA comprises a first Alu element and the second guide RNA comprises a second Alu element.

In certain embodiments, the first Alu element comprises an oligonucleotide sequence at least partially complementary to an oligonucleotide sequence in the second Alu element.

In some embodiments, the Alu element comprises an oligonucleotide sequence of from 50-250 nucleotides. In certain embodiments, the Alu element comprises an oligonucleotide sequence of from 50-250 nucleotides. In certain embodiments, the Alu element comprises an oligonucleotide sequence of about 225 nucleotides.

In certain embodiments, the ADAR recruiting domain comprises a GluR2 recruiting domain. In certain embodiments, the ADAR recruiting domain comprises an internal GluR2 loop and/or additional ADAR-recruiting dsRNA loops.

In certain embodiments, the internal GluR2 loop and/or additional ADAR-recruiting dsRNA loops permit targeting an adenosine in a pre-mRNA or mRNA sequence. In certain embodiments, the internal GluR2 loop comprises an asymmetric GluR split gRNA.

In certain embodiments, the ADAR recruiting domain has a structure that is capable of binding ADAR1, ADAR2, ADAR3, or any combination thereof.

In some embodiments, the adar-sgRNA comprises a 5′ RNA targeting domain on the first guide RNA. In some embodiments, the adar-sgRNA comprises a 3′ RNA targeting domain on the second guide RNA. In certain embodiments, the adar-sgRNA comprises both a 5′ RNA targeting domain on the first guide RNA and a 3′ RNA targeting domain on the second guide RNA.

In certain embodiments, the at least one RNA targeting domain comprises a plurality of mismatched ribonucleotides that are non-complementary to the sequence of the targeted segment of the target RNA.

In some embodiments, the at least one RNA targeting domain is from about 20 to about 100 nucleotides in length.

In some embodiments, the first or second guide RNA further comprises a modification at the 5′ and/or 3′ end.

In some embodiments, the target RNA is pre-mRNA, mRNA, non-coding RNA, or viral RNA.

In some embodiments, the target RNA comprises a mutation that results in altered or modified endogenous protein expression. In some embodiments, the mutation is a point mutation, optionally wherein the point mutation results in a missense mutation, splice site alteration, or a premature stop codon.

In some embodiments, the first or second guide RNA has a sequence selected from the group of sequences provided in FIG. 6.

In an aspect, the present disclosure provides a composition comprising at least one recombinant guide adar-sgRNA.

In another aspect, the present disclosure provides a vector, wherein the vector comprises a coding region, wherein the coding region encodes at least one recombinant guide adar-sgRNA encoding a first and second guide RNA. In certain embodiments, the guide adar-sgRNA coding region is operably linked to one or more expression control elements. In certain embodiments, the expression control elements and coding region are together flanked by 5′ and 3′ AAV terminal repeats (ITR).

In certain aspects, the vector is an AAV or lentiviral vector. In certain aspects, the AAV vector comprises AAV1, AAV2, AAV3, AAV4, AAV5, AAV6, AAV7, AAV8, AAV9, AAV10, AAV 11, AAV12, or any variants or chimeras thereof.

In certain embodiments, the vector is packaged into a virion.

In certain embodiments, the vector is formulated into a nanoparticle.

In another aspect, the disclosure provides a method for modifying the sequence of a target RNA, comprising contacting the target RNA with a recombinant guide adar-sgRNA, a composition, or a guide adar-sgRNA expressed from a vector as described herein. In some embodiments, the method further comprises delivering at least one polynucleotide encoding ADAR1, ADAR2, ADAR3, and/or any combination thereof. In some embodiments, the contacting is in vitro or in vivo. In certain embodiments, the vector is administered to a mammalian subject in whom editing of the target RNA is desired.

In another aspect, the disclosure provides a method of treating a disease or disorder resulting from the decrease or loss of wild-type expression of a protein, comprising delivering an effective amount of a recombinant guide adar-sgRNA, a composition, or a guide adar-sgRNA expressed from a vector as described herein, to cells of a patient having a disease or disorder resulting from the loss of wild-type expression of a protein, wherein the recombinant guide adar-sgRNA is formed by the first and second guide RNA bound to a RNA target in a trimolecular complex which reconstitutes the ADAR recruiting domain, thereby increasing expression of the protein whose expression is decreased or lost.

In some embodiments, the recombinant guide adar-sgRNA is delivered by administering a vector as described herein. In some embodiments, the target RNA encodes the protein whose expression is altered or modified compared to wild-type expression. In some embodiments, the subject is a human.

In an aspect, the disclosure further provides use of an effective amount of one or more recombinant guide adar-sgRNAs, a composition, or a guide adar-sgRNA expressed from a vector as provided herein, for treating a disease or disorder associated with loss of wild-type protein expression.

In an aspect, the disclosure also provides a kit comprising one or more recombinant guide adar-sgRNAs, a composition, or a guide adar-sgRNA expressed from a vector as provided herein, and, optionally, instructions for use.

In some embodiments, the recombinant guide adar-sgRNA disclosed herein is capable of hybridizing to a complementary target ribonucleotide under conditions of high stringency. In certain embodiments, the recombinant guide adar-sgRNA hybridizes to a complementary target ribonucleotide under conditions of high stringency equal to or greater than 37° C.

### 5. DETAILED DESCRIPTION OF THE DRAWINGS

FIG. 1 depicts active Alu elements residing in the POLR2A gene (AluSz, AluSx1).

FIG. 2 represents a schematic showing of Alu split-guide RNA designs of the present disclosure.

FIG. 3 represents a schematic showing of an Alu intact-guide RNA design of the present disclosure.

FIG. 4 describes alternative options for programmed RNA editing. The alternative options for programmed RNA editing are the Alu split and Alu intact (Internal to the guide) RNA guides. This figure highlights the editing efficiency of a target adenosine of the above guides.

FIG. 5 is a schematic showing an exemplary trimolecular complex of the present disclosure comprising a gRNA, a GluR2 hairpin internal to the guide RNA, and a target RNA hybridized to at least part of the gRNA and forming 3 A/C mismatches.

FIG. 6 describes asymmetric split gRNA decreases gRNA:gRNA Tm.

FIG. 7 represents a schematic showing a trimolecular complex of the present disclosure providing an aSHAPE guide RNA with improved thermodynamics.

FIG. 8 depicts predicted structures of split gRNA on and off a RAB7A target strand.

FIG. 9 represents a schematic showing two versions of split gRNA.

FIG. 10 describes structures of guide RNAs disclosed herein including split gRNAs, intact gRNAs, and linear gRNAs with no recruiting domain) that were cloned into an AAV plasmid backbone for various transfection and RNA editing experiments.

FIG. 11 illustrates RNA editing using various gRNAs disclosed herein.

FIG. 12 illustrates another exemplary aSHAPE editing on target RNA.

FIG. 13 describes effects of aSHAPE directionality on target gene expression.

FIG. 14 describes aSHAPE gRNA association restores editing to unbroken editing efficiency (referring to half-split editing efficiencies when compared to the full-split editing efficiency).

FIG. 15 illustrates potential directionality of aSHAPE, describing effects of aSHAPE directionality on percentage of a target gene editing by ddPCR.

### 6. DETAILED DESCRIPTION

We have developed a class of recombinant split RNA guide molecules modeled on mammalian RNA structures that are capable of recruiting Adenosine Deaminase acting on RNA (ADAR) enzymes to edit desired pre-mRNA and mRNA targets. The recombinant guide molecules are capable of recruiting native ADAR1, ADAR2, and/or ADAR3 enzymes to modify/change a specific adenosine to inosine in a target RNA.

Provided herein are classes of recombinant split RNA guide molecules modeled on mammalian RNA structures that are capable of recruiting Adenosine Deaminase acting on RNA (ADAR) enzymes to edit desired pre-mRNA and mRNA targets. Two exemplary approaches of a Split HAirpin for Programmed RNA Editing (SHAPE) are provided: 1) an Alu split guide RNA system (Alu-split gRNA) and 2) an aymmetric GluR split guide system (aSHAPE gRNA).] These approaches provide numerous benefits over other gene editing systems, including offering improved target binding selectivity, lower immunogenicity, and thermodynamics.

In certain embodiments, provided herein is a recombinant adenosine deaminase acting on RNA (ADAR) split guide RNA (adar-sgRNA), composition comprising two guides - one guide with an ADAR recruiting domain and a second guide with a 5′ and/or 3′ RNA targeting domain for forming a trimolecular complex at a mismatch site that serves to recruit ADAR to edit the site.

The Alu split-gRNA system will potentially circumvent the triggering of innate immune responses since two single split guides with minimal to no double-stranded character will be presented to cells (an Alu double stranded RNA hairpin structure is formed when the two guides achieve complementarity with a specific target) (see Quin J et al, ADAR RNA Modifications, the Epitranscriptome and Innate Immunity, Trends in Biochem Sci, In Press (2021)).

The Alu split-gRNA system will potentially circumvent aberrant RNA editing of endogenous targets. Double stranded RNAs formed by Alu elements actively recruit ADAR enzymes. In mammals, the most ubiquitous RNA editing occurs within these elements. Alu double stranded RNAs have the capacity to sequester endogenous ADAR enzymes, acting as “ADAR sinks”, when presented to cells leading to aberrant RNA editing of endogenous targets.

The Alu split-gRNA system will potentially circumvent ineffective RNA editing. When Alu elements are placed either at the 5′ or 3′ of a guide, RNA editing of the Alu double stranded RNA-target guide molecule can lead to overall cis-RNA structure destabilization. Unwanted cis-guide RNA mutations can lead to compromised target identification and inefficient RNA editing.

The Alu split-gRNA system will potentially improve AAV packaging. Incorporation of hairpins (dsRNAs) into rAAV constructs can reduce vector yield and can produces a population of truncated and defective genomes. The Alu split-gRNA system will potentially circumvent this issue, thus increasing AAV packaging efficiency.

### Asymetric Split HAirpin Editing (aSHAPE)

The aSHAPE gRNA design utilizes an internal GluR2 loop, or additional ADAR-recruiting dsRNA loops, and can be applied to any gRNA that is designed to target an adenosine in a pre-mRNA or mRNA sequence. Additionally, the GluR split gRNA offers improved thermodynamics, as described herein and shown in Example 1.

The ADAR editing system is tissue-specific. While the system can rely on endogenous ADAR expression to achieve the desired editing, concomitant delivery/expression of recombinant ADAR enzymes is also contemplated. Additionally, there is no editing of the genome when utilizing the ADAR system.

Unless described otherwise, all technical and scientific terms used herein have the meaning commonly understood by one of ordinary skill in the art to which the invention pertains.

As used herein, unless otherwise dictated by context “nucleotide” or “nt” refers to ribonucleotide.

As used herein, the terms “patient” and “subject” are used interchangeably, and may be taken to mean any living organism which may be treated with compounds of the present invention. As such, the terms “patient” and “subject” include, but are not limited to, any non-human mammal, primate and human.

A “therapeutically effective amount” of a composition is an amount sufficient to achieve a desired therapeutic effect and does not require cure or complete remission.

The terms “treat,” “treated,” “treating”, or “treatment” as used herein have the meanings commonly understood in the medical arts, and therefore does not require cure or complete remission, and therefore includes any beneficial or desired clinical results. Treatment includes eliciting a clinically significant response without excessive levels of side effects. Treatment also includes prolonging survival as compared to expected survival if not receiving treatment.

As used herein “preventing” a disease refers to inhibiting the full development of a disease.

The term “gRNA” or “guide RNA” as used herein refers to the guide RNA sequences used to target specific genes for correction employing the ADAR editing techniques (for a Review of ADAR editing See, Christofi and Zaravinos J. Transl. Med. 17:3 1 9, 2019). In some aspects, a gRNA is recombinant.

The terms “hairpin,” or “loop” is used in context of an oligonucleotide to refer to a structure formed in single stranded oligonucleotide when sequences within the single strand which are complementary when read in opposite directions base pair to form a region whose conformation resembles a hairpin or loop.

The term “target RNA” as used herein refers to mRNA and/or pre-mRNA.

The term “splint” as used herein refers to a molecule of target RNA that contains a 5′ sequence that is partially or fully complementary to the second guide RNA 3′ antisense sequence and a 3′ sequence that is partially or fully complementary to the first guide RNA 5′ antisense sequence.

The term “trimolecular” as used herein refers to the hybridization of three distinct nucleic acid molecules to each other thereby forming an intermolecular bound and stabilized molecular complex.

The term reconstituted, as used herein refers to the hybridization-mediated association of two RNA molecules to each other whereby the intermolecular association of the two RNA molecules establishes the natural and/or recreates the fully-intact RNA motif function.

### 6.1. Adar-Split gRNA

In a first aspect, recombinant adenosine deaminase acting on RNA (ADAR) split guide RNAs are provided (adar-split gRNA). The adar-split gRNA comprises an ADAR recruiting domain and at least one specific RNA targeting domain (complementary to only one target). The ADAR recruiting domain mimics the ADAR recruiting portion of a mammalian pre-mRNA, since the Alu elements are intronic. The RNA targeting domain is at the 5′ and/or 3′ end of the recruiting domain. At least one RNA targeting domain has a sequence that is only partially complementary to the sequence of segment of the target RNA. Binding of the two adar-split gRNAs to the target RNA forms a trimolecular complex which recruits ADAR enzymes to deaminate one or more mismatched adenosine residues in the adar-split gRNA RNA targeting domain:target RNA duplex.

### 6.1.1. ADAR Recruiting Domain

The ADAR recruiting domain of the recombinant adar-gRNA mimics certain aspects of the ADAR-recruiting portion of a mammalian RNA. The recruiting domain thus serves, in typical embodiments, to recruit ADAR 1, ADAR2, ADAR3, or any combination thereof, to the target sequence, and facilitates subsequent editing. In certain embodiments, the ADAR recruiting domain facilitates editing by ADAR1. In certain embodiments, the ADAR recruiting domain facilitates editing by ADAR2.

The ADAR recruiting domain comprises a dsRNA entity that is intact. In other embodiments the ADAR recruiting domain comprises two single-stranded RNAs (split-guides) that will form a dsRNA when both are transcribed within a cell at the same time. The ADAR recruiting domains such as the GluR2 and Alu- based guides do not contain double stranded binding domains or a deaminase domain. The ADAR enzymes contain said domains (the double stranded binding domains or deaminase domain, which in turn recognize the recruiting elements). In some embodiments, the ADAR recruiting domain comprises two single-stranded RNA-binding domains (ssRBDs) and a deaminase domain.

In various embodiments, the adar-split gRNA promotes both ADAR recruitment and target recognition by target-RNA hybridization. In some embodiments, site-directed RNA editing is achieved by guiding ADAR onto the target site.

In some embodiments, the ADAR recruiting domain binds target RNAs via a gRNA that hybridizes to the target RNA. In some embodiments, upon hybridization of a gRNA to the target RNA, where the gRNA is operably linked to an ADAR recruiting domain, the result is site-directed A-to-I editing of an adenosine in the target RNA by ADAR. In some embodiments, the site-directed A-to-I editing occurs without a modified deaminase.

In various embodiments, the recruiting domain is between 20-90 ribonucleotides in length. In some embodiments, the recruiting domain is between 50-80 ribonucleotides in length, or 60-70 ribonucleotides in length. In some embodiments, the recruiting domain is 45 nucleotides. In certain embodiments, the recruiting domain is 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt, 81 nt, 82 nt, 83 nt, 84 nt, 85 nt, 86 nt, 87 nt, 88 nt, 89 nt, or 90 nt in length.

In various embodiments, the ADAR recruiting domain comprises the ADAR-recruiting portion of a mammalian mRNA with one or more substitutions, insertions and/or deletions of nucleotides, so long as the ADAR recruiting activity is not lost. In some embodiments, the one or more substitutions, insertions and deletions of nucleotides improves a desired property of the recombinant adar-split gRNA.

In some embodiments, the ADAR recruiting domain is a GluR2 domain.

In some embodiments, the engineered/recombinant adar-split gRNA will recruit any one or a combination of ADAR1, ADAR2, and ADAR3. In some embodiments, the recombinant adar-split gRNA will have preferential binding to ADAR1. In some embodiments, the recombinant guide adar-split gRNA will have preferential binding to ADAR2. In some embodiments, the recombinant guide adar-split gRNA will have preferential binding to ADAR3.

The engineered guide recombinant adar-split gRNA can comprise a plurality of ADAR recruiting domains. In typical embodiments, the recombinant adar-split gRNA has one ADAR recruiting domain. In some embodiments, the recombinant adar-split gRNA has two ADAR recruiting domains.

### 6.1.2. RNA Targeting Domains of the Split Guide RNA

In typical embodiments, the sgRNA comprises an RNA targeting domain that is at least partially complementary to a target RNA. In some embodiments, the sgRNA comprises at least one RNA targeting domain having a sequence that is complementary to the sequence of segment of the target RNA. In some embodiments, the double stranded RNA duplex formed by a guide RNA and a target RNA comprises a mismatch at the target adenosine to be edited to an inosine. In some embodiments, the RNA targeting domain is an antisense oligonucleotide sequence.

### 6.1.3. Alu Split-Guide Elements

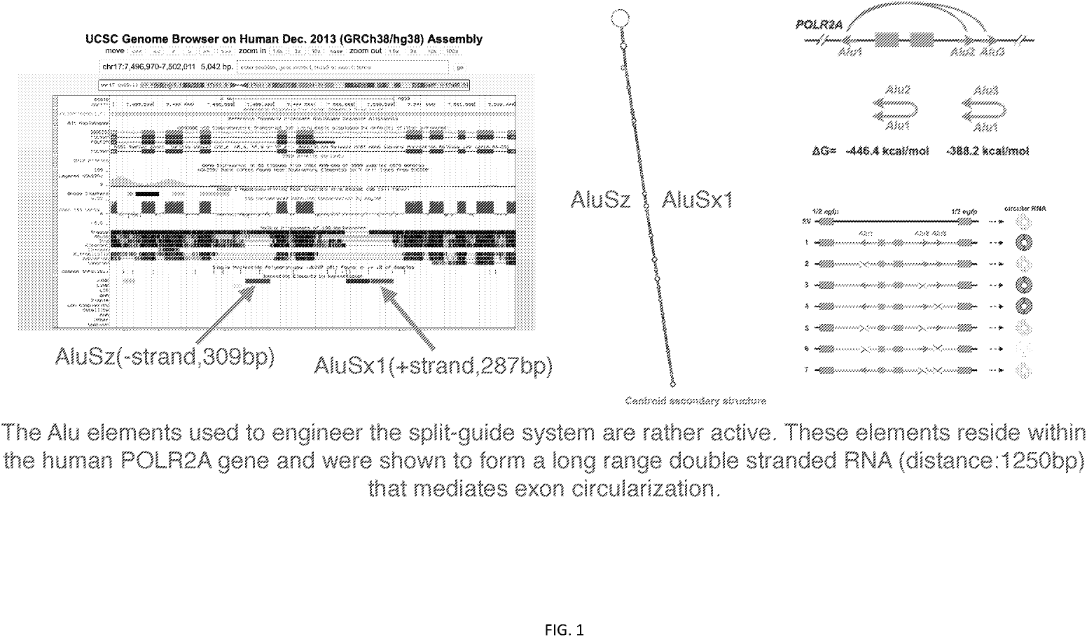

In various embodiments, the sgRNA comprises an antisense oligonucleotide sequence comprising a segment of an Alu element. In some embodiments, the Alu element comprises a segment of an oligonucleotide from the human POLAR2A gene. FIG. 1 shows the native sequences of an Alu element from the human POLR2A gene that forms a long-range double stranded RNA (distance: 1250bp, which is the nucleotide distance between the two natural Alu elements with the POLR2A gene) that mediates exon circularization.

In various embodiments, the sgRNA comprises a first guide and a second guide. In some embodiments, the first guide comprises a first 5′ Alu segment and the second guide comprises a second 3′ Alu segment. In some embodiments, the first guide comprising the 5′ Alu segment comprises an oligonucleotide sequence at least partially complementary to an oligonucleotide sequence in the second guide comprising the 3′ Alu element.

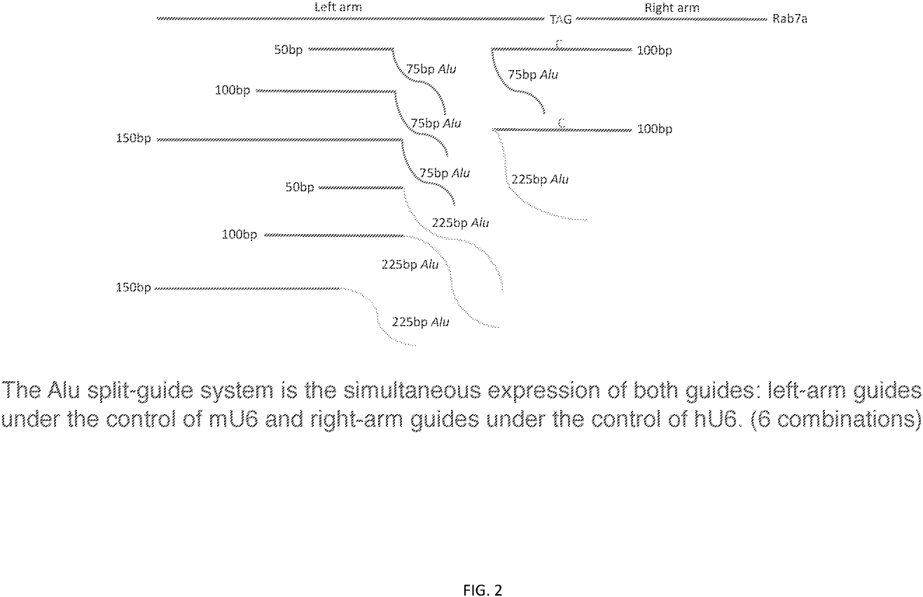

In various embodiments, the Alu element comprises a 5′ segment comprising an antisense nucleotide sequence having no complementary nucleotides with the nucleotide sequence of the target RNA (FIG. 2). In various embodiments, the Alu element comprises a 3′ segment comprising an antisense nucleotide sequence having complementary with at least one nucleotide of the target RNA. In some embodiments, the antisense nucleotide sequence comprises an oligonucleotide sequence complementary to the target RNA. In some embodiments, the complementary sequence of the Alu element binds to the complementary nucleotide on target RNA, thereby promoting RNA editing.

In various embodiments, the Alu element is expressed under the control of at least one promoter. In some embodiments, the promoter is a human U6 promoter. In some embodiments, the promoter is a mouse U6 promoter.

In various embodiments, the Alu split-guide system is the simultaneous expression of both guides wherein the first split guide RNA which contains the 5′ Alu segment and optional 5′ antisense domain is under the control of a mU6 promoter and second split guide RNA which contains the 3′ Alu segment and optional 3′ antisense domain is under the control of a hU6 promoter. In some embodiments, the Alu Intact-guides are under the control of a hU6 promoter. In some embodiments, a promoter that may be used with any of the guide RNAs disclosed herein may be a U6 promoter, a U7 promoter, a U1 promoter, a 7SK promoter, a H1 promoter, and any variants or truncations of any of these promoters.

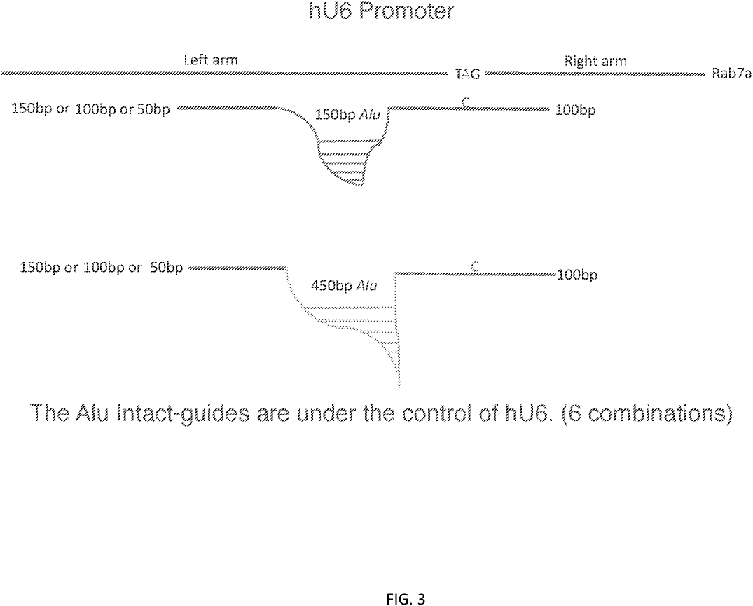

In various embodiments, the sgRNA comprises an Alu segment that is between 100-500 ribonucleotides in length (FIG. 3). In some embodiments, the Alu element is between 125-450 ribonucleotides in length, or 150-375 ribonucleotides in length, 200-300 ribonucleotides in length. In certain embodiments, the Alu element is 100 nt, 125 nt, 150 nt, 175 nt, 200 nt, 225 nt, 250 nt, 275 nt, 300 nt, 325 nt, 350 nt, 375 nt, 400 nt, 425 nt, 450 nt, or 500 nt in length. In some embodiments, the Alu element is 150 nt. In some embodiments, the Alu element is 300 nt. In some embodiments, the Alu element is 450 nt.

In various embodiments, the first guide RNA is between 100-400 ribonucleotides in length. In some embodiments, the first guide RNA is between 125-375 ribonucleotides in length, or 175-325 ribonucleotides in length, 225-275 ribonucleotides in length. In certain embodiments, the first guide RNA is 100 nt, 125 nt, 150 nt, 175 nt, 200 nt, 225 nt, 250 nt, 275 nt, 300 nt, 325 nt, 350 nt, 375 nt, or 400 nt in length.

In some embodiments, the first guide RNA comprises an antisense nucleotide sequence having between 20-200 ribonucleotides in length. In some embodiments, the antisense nucleotide sequence is between 30-180 ribonucleotides in length, or 50-100 ribonucleotides in length, 100-150 ribonucleotides in length. In certain embodiments, the antisense nucleotide sequence is 30 nt, 50 nt, 75 nt, 100 nt, 125 nt, 150 nt, 175 nt, or 200 nt in length. In some embodiments, the carrier nucleotide sequence is 50 nt. In some embodiments, the antisense nucleotide sequence is 100 nt. In some embodiments, the antisense nucleotide sequence is 150 nt.

In some embodiments, the first guide RNA comprises an Alu element having between 20-250 ribonucleotides in length. In some embodiments, the Alu element is between 50-250 ribonucleotides in length, or 75-200 ribonucleotides in length, 125-225 ribonucleotides in length. In certain embodiments, the Alu element is 20 nt, 50 nt, 75 nt, 100 nt, 125 nt, 150 nt, 175 nt, 200 nt, 225 nt, or 250 nt in length. In some embodiments, the first guide RNA comprises an Alu element having about 75 nt. In some embodiments, the first guide RNA comprises an Alu element having about 150 nt. In some embodiments, the first guide RNA comprises an Alu element having about 225 nt.

In various embodiments, the second guide RNA is from 50-400 ribonucleotides in length. In some embodiments, the second guide RNA is from 100-350 ribonucleotides in length, or 150-325 ribonucleotides in length, 175-300 ribonucleotides in length. In certain embodiments, the second guide RNA is 50 nt, 75 nt, 100 nt, 125 nt, 150 nt, 175 nt, 200 nt, 225 nt, 250 nt, 275 nt, 300 nt, 325 nt, 350 nt, 375 nt, or 400 nt in length.

In some embodiments, the second guide RNA comprises a antisense nucleotide sequence having from 20-200 ribonucleotides in length. In some embodiments, the antisense nucleotide sequence is from 30-180 ribonucleotides in length, or 50-100 ribonucleotides in length, 100-150 ribonucleotides in length. In certain embodiments, the antisense nucleotide sequence is 30 nt, 50 nt, 75 nt, 100 nt, 125 nt, 150 nt, 175 nt, or 200 nt in length. In some embodiments, the antisense nucleotide sequence is 50 nt. In some embodiments, the antisense nucleotide sequence is 100 nt. In some embodiments, the antisense nucleotide sequence is 150 nt.

In some embodiments, the second guide RNA comprises an Alu element having from 20-250 ribonucleotides in length. In some embodiments, the Alu element is from 50-250 ribonucleotides in length, or 75-200 ribonucleotides in length, 125-225 ribonucleotides in length. In certain embodiments, the Alu element is 20 nt, 50 nt, 75 nt, 100 nt, 125 nt, 150 nt, 175 nt, 200 nt, 225 nt, or 250 nt in length. In some embodiments, the second guide RNA comprises an Alu element having about 75 nt. In some embodiments, the second guide RNA comprises an Alu element having about 150 nt. In some embodiments, the second guide RNA comprises an Alu element having about 225 nt.

### 6.2. Asymmetric Split Hairpin Editing (aSHAPE)

In a second aspect, asymmetric split GluR2 hairpin editing (aSHAPE) guide RNAs are provided. The aSHAPE comprises an internal GluR2 hairpin and at least one antisense domain that is at least partially complementary to a target RNA. The antisense region of the gRNA is modified to recognize the target pre-mRNA or mRNA sequence. The internal GluR2 hairpin is split into two asymmetric 5′ and 3′ segments, with the 5′ GluR2 segment located within the first guide RNA and the 3′ GluR2 segment located within the second guide RNA. The first and second guide RNAs have at least partial complementary to each other. Upon hybridization of the first and second guide RNAs to the target RNA, the GluR2 hairpin is re-constituted. The binding of the first guide RNA and the second guide RNA to the target RNA, thus, forms a trimolecular complex, which contains a reconstituted GluR2 hairpin capable of recruiting ADAR for target-specific RNA-editing.

### 6.2.1. GluR2 Split Hairpin

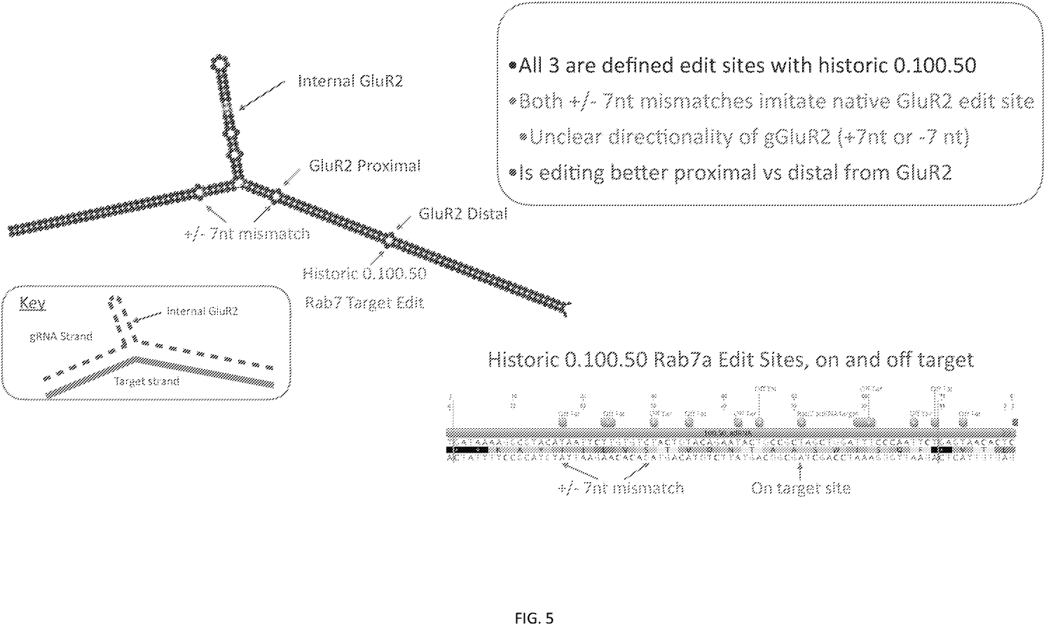

In typical embodiments, the aSHAPE system comprises an internal GluR2 hairpin, two GluR2 proximal A/C mismatch and a GluR2 distal A/C mismatch (FIG. 5).

In various embodiments, the GluR2 proximal A/C mismatch comprises a +/- 7 nt mismatch imitating native GluR2 edit site.

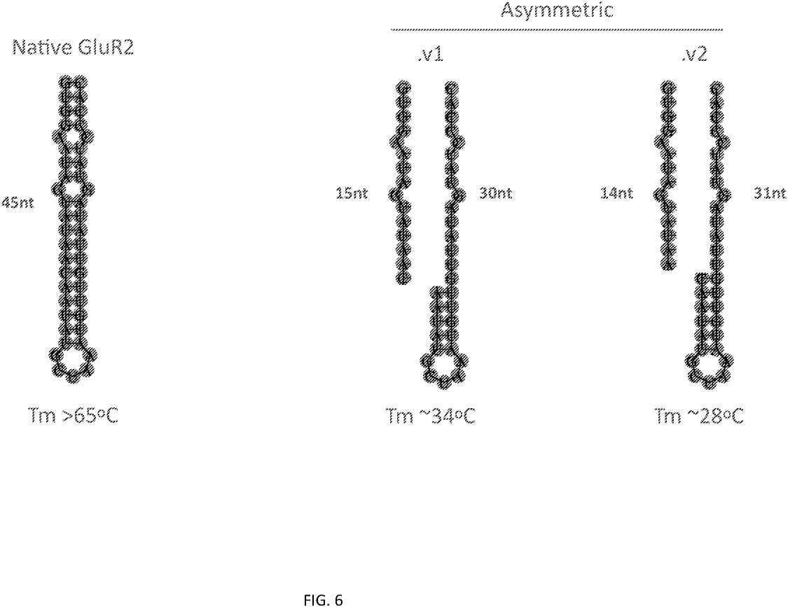

In various embodiments, the aSHAPE system comprises an asymmetric GluR2 hairpin (FIG. 6). In some embodiments, the intact GluR2 comprises 45 nt. In some embodiments, the GluR2 hairpin is split into two asymmetric fragments. For example, the GluR2 hairpin is split into a fragment of 15 nt and a fragment of 30 nt. As another example, the GluR2 hairpin is split into a fragment of 14 nt and a fragment of 31 nt. In some embodiments, the intact GluR2 has the sequence of SEQ ID NO: 1. In some embodiments, the 15 nt fragment has the sequence of SEQ ID NO:2. In some embodiments, the 30 nt fragment has the sequence of SEQ ID NO:3. In some embodiments, the 14 nt fragment has the sequence of SEQ ID NO:4. In some embodiments, the 31 nt fragment has the sequence of SEQ ID NO: 5.

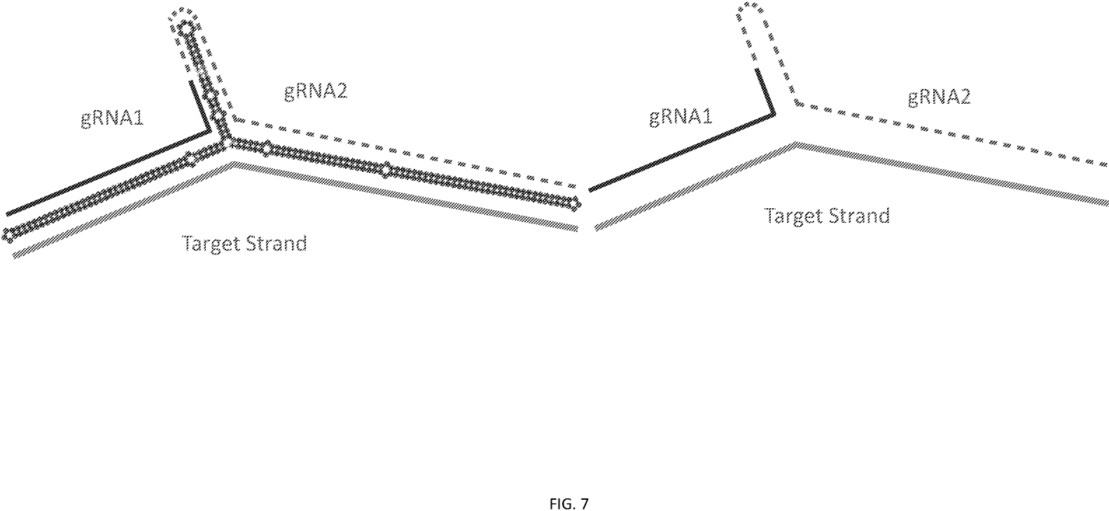

In various embodiments, the aSHAPE system comprises a first gRNA strand (gRNA1; version 1) and a second gRNA strand (gRNA2; version 2) (FIG. 7). In some embodiments, gRNA1 is at least partially complementary to a target RNA. In some embodiments, gRNA1 is at least complementary to gRNA2. In some embodiments, gRNA2 is at least partially complementary to a target RNA. In some embodiments, gRNA2 is at least complementary to gRNA1.

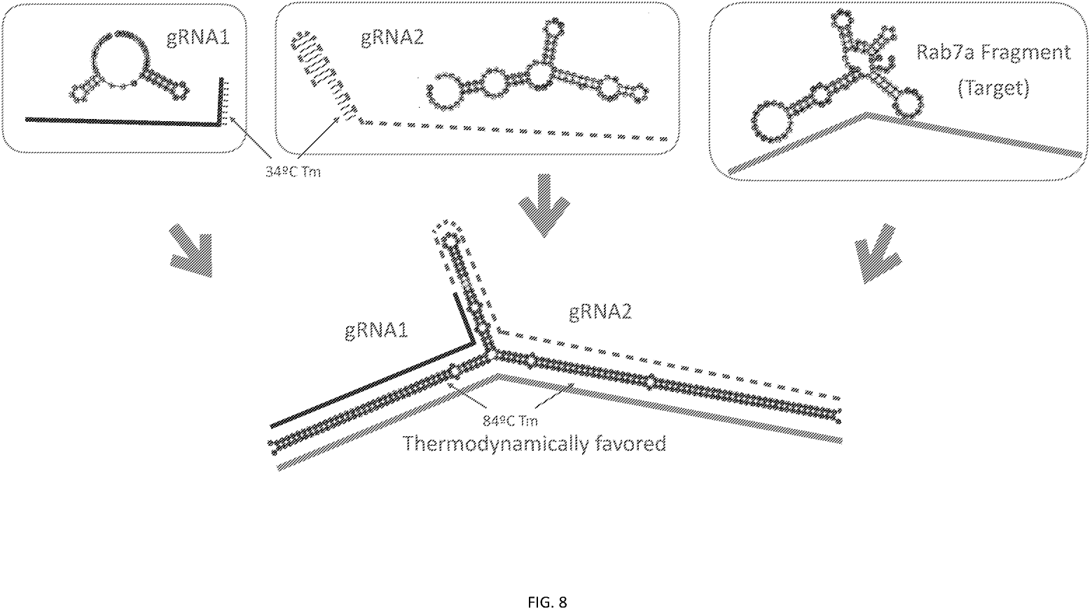

In some embodiments, gRNA1 and gRNA2 permit decrease gRNA:gRNA Tm. In some embodiments, gRNA1 has a lower Tm than native GluR2. In some embodiments, gRNA2 has lower Tm than native GluR2. In some embodiments, association of the gRNA1 and gRNA2 forms a functional GluR2 hairpin; thereby improving thermodynamics of the gRNA:target complex (FIG. 8).

In various embodiments, gRNA1 is from 1-45 ribonucleotides in length. In some embodiments, gRNA1 is from 5-15 ribonucleotides in length, or 10-20 ribonucleotides in length, or 12-16 ribonucleotides in length. In certain embodiments, gRNA1 is 5 nt, 10 nt, 12 nt, 13 nt, 14 nt, 15 nt, 16 nt, 17 nt, 18 nt, 19 nt 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt or 44 nt in length. In some embodiments, gRNA1 is 14 ribonucleotides in length. In some embodiments, gRNA1 is 15 ribonucleotides in length.

In various embodiments, gRNA2 is from 1-45 ribonucleotides in length. In some embodiments, gRNA1 is from 5-15 ribonucleotides in length, or 10-20 ribonucleotides in length, or 12-16 ribonucleotides in length. In certain embodiments, gRNA2 is 5 nt, 10 nt, 12 nt, 13 nt, 14 nt, 15 nt, 16 nt, 17 nt, 18 nt, 19 nt 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt or 44 nt in length. In some embodiments, gRNA2 is 30 ribonucleotides in length. In some embodiments, gRNA2 is 31 ribonucleotides in length.

In various embodiments, the aSHAPE system has a Tm from 10-100° C. In some embodiments, the aSHAPE system has a Tm between a Tm from 20-80° C., or 30-70° C., 25-40° C., 20-30° C., 25-35° C. In certain embodiments, the aSHAPE system has a Tm at 10° C., 20° C., 22° C., 25° C., 28° C., 30° C., 32° C., 34° C., 40° C., 45° C., 50° C., 55° C., 60° C., 65° C., 70° C., 75° C., 80° C., 85° C., 90° C., 95° C., or 100° C.

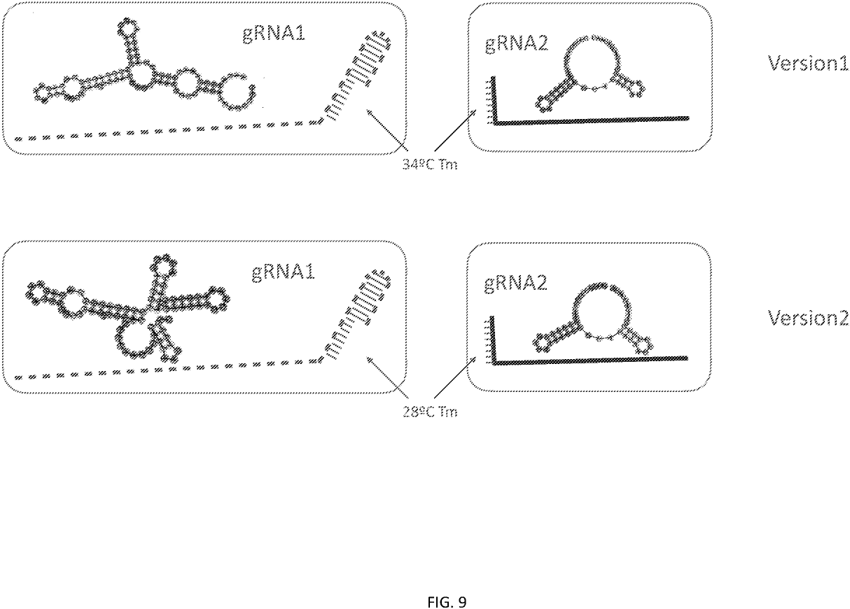

In various embodiments, gRNA1 has a Tm from 10-50° C. (FIG. 9). In some embodiments, gRNA1 at a Tm from 15-45° C., or 20-30° C., 25-35° C., 30-40° C. In certain embodiments, gRNA1 has a Tm at 15° C., 18° C., 20° C., 22° C., 24° C., 26° C., 28° C., 30° C., 30° C., 32° C., 34° C., 36° C., 38° C., 40° C., 45° C., or 50° C. In some embodiments, gRNA1 has a Tm at 34° C.

In various embodiments, gRNA2 has a Tm from 10-50° C. (FIG. 9). In some embodiments, gRNA2 has a Tm from 15-45° C., or 20-30° C., 25-35° C., 30-40° C. In certain embodiments, gRNA2 has a Tm at 15° C., 18° C., 20° C., 22° C., 24° C., 26° C., 28° C., 30° C., 30° C., 32° C., 34° C., 36° C., 38° C., 40° C., 45° C., or 50° C. In some embodiments, gRNA2 has a Tm at 28° C.

In various embodiments, the gRNA:target complex has a Tm from 10-100° C. In some embodiments, gRNA1 at a Tm from 20-80° C., or 30-70° C., 25-40° C., 20-30° C., 25-35° C. In certain embodiments, the gRNA:target complex has a Tm at 10° C., 20° C., 22° C., 25° C., 28° C., 30° C., 32° C., 34° C., 40° C., 45° C., 50° C., 55° C., 60° C., 65° C., 70° C., 75° C., 80° C., 85° C., 90° C., 95° C., or 100° C. In some embodiments, the complex has Tm at 84° C.

The recombinant adar-split gRNA comprises at least one RNA targeting domain that localizes the recombinant adar-split gRNA to the target RNA desired to be edited through sequence complementarity to a segment of the target RNA. Thus, the RNA targeting domain has a sequence that is sufficiently “antisense” to the sequence of the target RNA to permit hybridization of the RNA targeting domain to the target RNA. At least one of the at least one RNA targeting domains has a sequence that is only partially complementary to the sequence of the segment of the target RNA desired to be edited.

In some embodiments, the recombinant adar-split gRNA comprises one RNA targeting domain. The RNA targeting domain is only partially complementary to the sequence of the segment of the target RNA desired to be edited. In certain embodiments, the RNA targeting domain is at the 5′ end of the recruiting domain. In certain embodiments, the RNA targeting domain is at the 3′ end of the recruiting domain.

In some embodiments, the recombinant adar-split gRNA comprises two RNA targeting domains, one at the 5′ end of the recruiting domain and one at the 3′ end of the recruiting domain. In some embodiments, one of the two RNA targeting domains is only partially complementary to the sequence of the segment of the target RNA desired to be edited, and the other RNA targeting domain is fully complementary in sequence to the target RNA. In some embodiments, both of the RNA targeting domains are partially complementary to the sequence of the target RNA, each to a segment of the target RNA desired to be edited.

In various embodiments, the partially complementary RNA targeting domain has only 1 mismatched nucleotide that is non-complementary to the sequence of the targeted segment of the target RNA. In various embodiments, the partially complementary RNA targeting domain has a plurality of mismatched nucleotides that are non-complementary to the sequence of the targeted segment of the target RNA.

In embodiments with a single RNA targeting domain, the RNA targeting domain is of sufficient length to bind to the target RNA under intracellular conditions, notwithstanding the one or more complementarity mismatches. In preferred embodiments, the RNA targeting domain is of sufficient length to bind under intracellular conditions preferentially to the target RNA over RNA species that are not desired to be edited. In embodiments with two RNA targeting domains, the RNA targeting domains are together of sufficient length to bind to the target RNA under intracellular conditions, notwithstanding the one or more complementarity mismatches in one or both RNA targeting regions. In preferred embodiments, the RNA targeting domains are together of sufficient length to bind under intracellular conditions preferentially to the target RNA over RNA species that are not desired to be edited.

In typical embodiments, the RNA targeting domain is from about 20 to about 100 nucleotides in length. In some embodiments, the RNA targeting domain is from 20-100 nt, 30-90 nt, 40-80 nt, or 50-70 nt in length. In some embodiments, the RNA target domain is at least 20, 25, 30, 35, 40, 45, 50, 55, 60, 65, 70, 75, 80, 85 or 90 nucleotides in length. In some embodiments, the RNA targeting domain is no more than 90, 80, 70, 60, 50, 40, or 30 nucleotides in length.

### 6.2.2. Target RNA

In some embodiments, the target RNA to be edited is a pre-mRNA. In some embodiments, the target RNA is a mature mRNA. In some embodiments, the target RNA is a miRNA or siRNA.

In certain embodiments, the target RNA is a splice acceptor or donor site. In certain additional embodiments, the target RNA is a transcriptional start site.

In currently preferred embodiments, the target RNA is an mRNA and/or pre-mRNA. In some embodiments, the mRNA and/or pre-mRNA comprises a mutation that results in loss of wild-type protein expression, and editing effected by contacting the target RNA with the recombinant adar-split gRNA increases expression of the protein encoded by the RNA. In some embodiments, full expression of the protein is restored. In some embodiments, partial expression is restored. In particular embodiments, sufficient expression is restored to improve signs or symptoms of a disease or disorder. In select embodiments, the target RNA is expressed from a mutated gene that causes one of the diseases described in section 4.8 below.

In certain embodiments, the target RNA comprises a point mutation. In particular embodiments, the point mutation results in a missense mutation, splice site alteration, or a premature stop codon.

In some embodiments, the recombinant adar-split gRNAs disclosed herein introduce mutations into a target RNA, where the mutations are introduced in order to prevent disease pathogenesis.

### 6.2.3. Other Modifications

In some embodiments, the recombinant adar-split gRNA further comprises a modification at the 5′ or 3′ end. In certain embodiments, the modification reduces exonuclease digestion of the recombinant adar-split gRNA.

In some embodiments the recombinant adar-split gRNA comprises one or more non-natural nucleotides. In some embodiments, the recombinant adar-split gRNA includes inter-nucleotide linkages that are not phosphodiester linkages.

### 6.3. Compositions Comprising Adar-Split gRNA

The recombinant guide nucleic acid molecules described herein can be in any number of suitable forms, including in naked form, in complexed form, or in a delivery vehicle.

In certain embodiments, the recombinant adar-split gRNA guide is in naked form. In particular embodiments, the recombinant adar-split gRNA is in a fluid composition without any other carrier proteins or delivery vehicles. In certain embodiments, the recombinant adar-split gRNA is in complexed form, bound to other nucleic acid or amino acids that assist in maintaining stability, such as by reducing exonuclease or endonuclease digestion.

In some embodiments, the recombinant adar-split gRNA is formulated into a composition that comprises the recombinant adar-split gRNA and at least one carrier or excipient. In some embodiments intended for direct administration of the recombinant adar-split gRNA to a patient, the recombinant adar-split gRNA is formulated in a pharmaceutical composition that comprises the recombinant adar-split gRNA and at least one pharmaceutically acceptable carrier or excipient. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can further be incorporated into the compositions.

Delivery vehicles such as liposomes, nanocapsules, microparticles, microspheres, lipid particles, vesicles, and the like, may be used for the introduction of any of the recombinant nucleic acids or compositions described herein into suitable host cells. In particular, the compositions or recombinant nucleic acids may be formulated for delivery either encapsulated in a lipid particle, a liposome, a vesicle, a nanosphere, or a nanoparticle or the like.

Methods to deliver recombinant guide nucleic acid molecules and related compositions described herein include any suitable method including: via nanoparticles include using liposomes, synthetic polymeric materials, naturally occurring polymers and inorganic materials to form nanoparticles.

Examples of lipid-based materials for delivery of the DNA or RNA molecules include: polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N-(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., ceDNA) can also be complexed with, e.g., poly (L-lysine) or avidin and lipids may, or may not, be included in this mixture, e.g., steryl-poly (L-lysine).

Naturally occurring polymers which can be used include chitosan, protamine, atelocollagen and peptides.

Inorganic materials that may also be used include gold nanoparticles, silica-based, and magnetic nanoparticles, which may be produced by methods known to the person skilled in the art.

### 6.4. Vectors Encoding Adar-Split gRNA

In another aspect, vectors encoding a recombinant adar-split gRNA are provided.

The adar-split gRNA is comprised of a first guide RNA and a second guide RNA. The first guide RNA is comprised of a 5′ segment of the ADAR recruiting domain, a 5′ RNA antisense targeting domain, or both domains. The second guide RNA is comprised of a 3′ segment of the ADAR recruiting domain, a 3′ RNA antisense targeting domain, or both domains. At least one of the 5′ or 3′ antisense RNA targeting domains is partially complementary to the target RNA. Binding of the first guide RNA and the second guide RNA to the target RNA forms a trimolecular hybridized complex at a 1:1:1 ratio. Moreover, the target RNA forms a molecular splint between the first and second guide RNAs. Formation of the target splint mediates reconstitution of the ADAR recruiting domain by colocalizing the 5′ segment of the ADAR recruiting domain and the 3′ segment of the ADAR recruiting domain, respective to the first and second guide RNA.

In some embodiments, the vector does not express the adar-split gRNA and is used to propagate polynucleotides that encode the adar-split gRNA. In typical embodiments, the encoding polynucleotide is DNA. In some embodiments, the vector is a plasmid. In some embodiments, the vector is a phage. In some embodiments, the vector is a phagemid. In some embodiments, the vector is a cosmid.

In some embodiments, the vector is capable of expressing the adar-split gRNA. Expression vectors can be used to introduce the recombinant adar-split gRNA into cells in vitro or in vivo.

In typical expression vector embodiments, the vector comprises a coding region, wherein the coding region encodes at least one recombinant adar-split gRNA as described herein. The coding region is operably linked to expression control elements that direct transcription. In some embodiments, the expression vector is an adenoviral vector, an adeno-associated virus (AAV) vector, a retroviral vector, or a lentiviral vector. In certain preferred embodiments, the vector is an AAV vector, and the expression control elements and adar-split gRNA coding region are together flanked by 5′ and 3′ AAV inverted terminal repeats (ITR).

In some examples, the vector may be a eukaryotic vector, a prokaryotic vector (e.g., a bacterial vector) a viral vector, or any combination thereof. In some examples, the vector may be a viral vector. In some examples, the viral vector may be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some examples, the viral vector may be a recombinant vector, a hybrid vector, a chimeric vector, a self-complementary vector, a single-stranded vector, or any combination thereof.

In some examples, the viral vector may be an adeno-associated virus (AAV). In some examples, the AAV may be any known AAV. In some examples, the viral vector may be of a specific serotype. In some examples, the viral vector may be an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV 10 serotype, an AAV 11 serotype, an AAV12 serotype, a derivative of any of these, or any combination thereof.

In some examples, the AAV vector may be a recombinant vector, a hybrid AAV vector, a chimeric AAV vector, a self-complementary AAV (scAAV) vector, a single-stranded AAV, or any combination thereof.

In some examples, a hybrid AAV vector may be produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV5 or AAV9), wherein the first and second AAV serotypes may be not be the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector may be a chimeric AAV vector. In some examples, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the AAV vector comprises a self-complementary AAV genome. Self-complementary AAV genomes may be generally known in the art and contain both DNA strands which can anneal together to form double-stranded DNA.

In some examples, the delivery vector may be a retroviral vector. In some examples, the retroviral vector may be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector may be transfected such that the majority of sequences coding for the structural genes of the virus (e.g., gag, pol, and env) may be deleted and replaced by the gene(s) of interest.

In some embodiments, the vector is packaged into a recombinant virion. In particular embodiments, the vector is packaged into a recombinant AAV virion.

### 6.5. Compositions Comprising Adar-Split gRNA-Encoding Vectors

In another aspect, compositions comprising the adar-split gRNA-encoding vectors are provided.

In some embodiments, the compositions are suitable for administration to a patient, and the composition is a pharmaceutical composition comprising a recombinant virion and at least one pharmaceutically acceptable carrier or excipient. In typical embodiments, the pharmaceutical composition is adapted for parenteral administration. In certain embodiments, the pharmaceutical composition is adapted for intravenous administration, intravitreal administration, posterior retinal administration, intrathecal administration, or intra-cisterna magna (ICM) administration. The administering can be a parenchymal injection, an intraventricular injection, an intra-cisternal injection, an intramuscular injection, intracerbroventricular (ICV) administration, subcutaneous injection, oral administration, mucosal administration, sublingual administration, buccal administration, rectal administration, ocular administration, otic administration, nasal administration, topical administration, cutaneous administration, transdermal administration, or any combination thereof.

### 6.6. Methods for Editing RNA

To effect editing of RNA, the adar-split gRNA is contacted to the target RNA in the presence of endogenous ADAR enzymes. Typically, contact is within a cell. In certain embodiments, the contacting is performed in vitro. In certain embodiments, the contacting is performed in vivo. In some embodiments, to effect editing of RNA, the adar-split gRNA is contacted to the target RNA in the presence of exogenously delivered ADAR enzymes. The ADAR enzyme is human ADAR1, human ADAR2, human ADAR3, or any combination thereof.

The adar-split gRNA is comprised of a first guide RNA and a second guide RNA. The first guide RNA is comprised of a 5′ segment of the ADAR recruiting domain, a 5′ RNA antisense targeting domain, or both domains. The second guide RNA is comprised of a 3′ segment of the ADAR recruiting domain, a 3′ RNA antisense targeting domain, or both domains. At least one of the 5′ or 3′ antisense RNA targeting domains is partially complementary to the target RNA. Binding of the first guide RNA and the second guide RNA to the target RNA forms a trimolecular hybridized complex at a 1:1:1 ratio. Moreover, the target RNA forms a molecular splint between the first and second guide RNAs. Formation of the target splint mediates reconstitution of the ADAR recruiting domain by colocalizing the 5′ segment of the ADAR recruiting domain and the 3′ segment of the ADAR recruiting domain, respective to the first and second guide RNA.

Thus, in another aspect, methods are provided for editing target RNAs. The methods comprise contacting the target RNA with at least one recombinant guide adar-split gRNA as described herein. In some embodiments, the contacting is performed in vitro. In some embodiments, the contacting is performed in vivo.

In some embodiments, the method comprises the preceding step of introducing one or more recombinant adar-split gRNAs into a cell comprising the target RNA. In some embodiments, the method comprises the preceding step of introducing one or more recombinant expression vectors that are capable of expressing the one or more recombinant adar-split gRNAs into the cell. In some embodiments, the methods further comprise delivering an ADAR enzyme, or ADAR-encoding polynucleotide, into the cell.

The recombinant guide nucleic acid molecules and vectors disclosed herein can be introduced into desired or target cells by any techniques known in the art, such as liposomal transfection, chemical transfection, micro-injection, electroporation, gene-gun penetration, viral infection or transduction, transposon insertion, jumping gene insertion, or any combination thereof.

The recombinant guide nucleic acid molecules and related compositions disclosed herein can be delivered by any suitable system, including by using any gene delivery vectors, such as adenoviral vector, adeno-associated vector, retroviral vector, lentiviral vector, or a combination thereof. In some embodiments, a recombinant adenoviral vector, a recombinant adeno-associated vector, a recombinant retroviral vector, a recombinant lentiviral vector, or a combination thereof, may be used to introduce any of the recombinant guide molecules or nucleic acid molecules described herein.

In some embodiments, the recombinant guide nucleic acid molecules disclosed herein may be present in a composition comprising physiologically acceptable carriers, excipients, adjuvants, or diluents. Neutral buffered saline or saline mixed with serum albumin are exemplary appropriate diluents. Suitable carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The pharmaceutically acceptable carriers (vehicles) useful in this disclosure are conventional. In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, compositions, whether they be solutions, suspensions or other like form, may include one or more of the following: DMSO, sterile diluents such as water for injection, saline solution, preferably physiological saline, Ringer’s solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

### 6.7. Methods of Treating Diseases Caused Altered Wild-Type Protein Expression

In another aspect, methods are provided for treating diseases caused by loss of wild-type expression. The method comprises delivering an effective amount of at least one recombinant guide adar-split RNA to a patient having a disease or disorder resulting from the loss of wild-type expression of a protein, wherein the recombinant guide adar-split RNA is capable of recruiting ADAR to edit an RNA target, thereby increasing expression of the protein whose expression is decreased or lost.

In another aspect, methods are provided for treating diseases caused by gain of wild-type expression. The method comprises delivering an effective amount of at least one recombinant guide adar-split RNA to a patient having a disease or disorder resulting from the gain of wild-type expression of a protein, wherein the recombinant guide adar-split RNA is capable of recruiting ADAR to edit an RNA target, thereby decreasing expression of the protein whose expression is aberrant.

There are numerous examples of diseases or conditions caused by aberrant protein expression, ( either increased or decreased from wild-type expression) that would be suitable for treatment using the methods described herein relating to ADAR editing.

An example includes conditions caused by missense mutations, which can render the resulting protein nonfunctional. Examples of such mutations can be responsible for human diseases including Epidermolysis bullosa, sickle-cell disease, Factor 5 Leiden, Rett Syndrome, Duchenne muscular dystrophy, and SOD1 mediated amyotrophic lateral sclerosis (ALS), Alzheimer’s disease, Parkinson’s disease, any genetic neurological disease or condition, or any genetic disease or condition. Another example is cystic fibrosis (Human Molecular Genetics, Vol.7, Issue 11, October 1998, Pages 1761-1769.).

The RNA editing techniques and methods described herein are likely to be most beneficial during the newborn or infant stages, but may provide benefits at any stage of life. The term pediatric typically refers to anyone under 15 years of age, and less than 35 kg. A neonate typically refers to newborn up to first 28 days of life. The term infant typically refers to an individual from the neonatal period up to 12 months. The term toddler typically refers to an individual from 1-3 years of age. Teenagers are typically considered to be 13-19 years of age. Young adults are typically considered to be from 19-24 years of age.

### 6.8. Kits

Additionally, certain components or embodiments of the heterologous/recombinant engineered guide nucleic acid molecule compositions can be provided in a kit. For example, any of the heterologous/recombinant engineered guide nucleic acid compositions, as well as the related buffers or other components related to administration can be provided frozen and packaged as a kit, alone or along with separate containers of any of the other agents from the pre-conditioning or post-conditioning steps, and optional instructions for use. In some embodiments, the kit may comprise ampoules, disposable syringes, capsules, vials, tubes, or the like. In some embodiments, the kit may comprise a single dose container or multiple dose containers comprising the embodiments herein. In some embodiments, each dose container may contain one or more unit doses. In some embodiments, the kit may include an applicator. In some embodiments, the kits include all components needed for the stages of conditioning/treatment. In some embodiments, the compositions may have preservatives or be preservative-free (for example, in a single-use container).

### 6.9. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### EXAMPLE 1

The following experiments further delineate key aspects of the adar-split gRNAs.

### A Alu Split-gRNA System

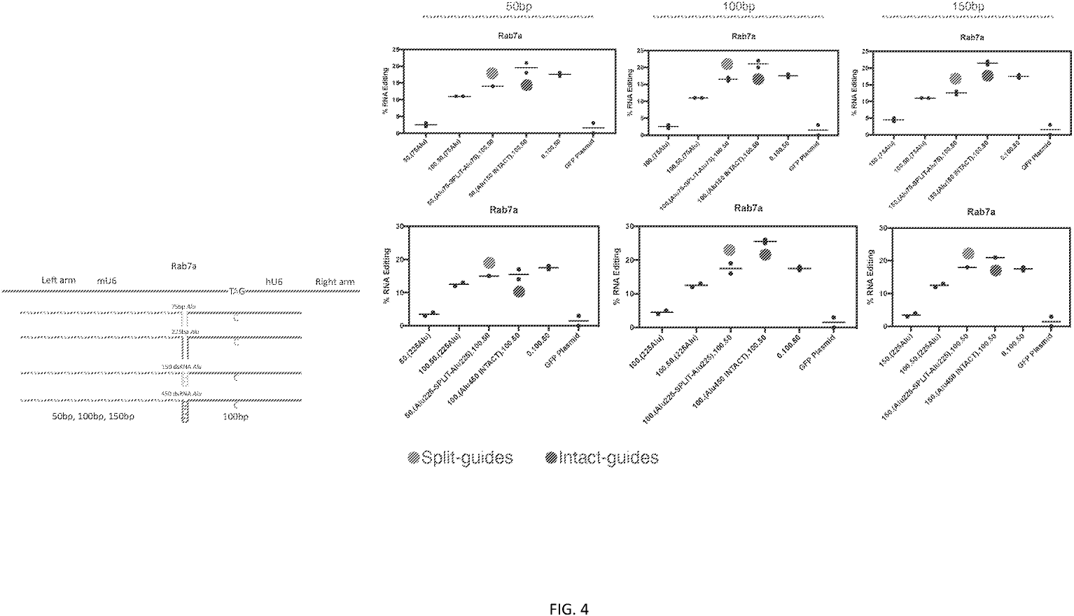

Experiments were performed to confirm the Alu split-gRNA system RNA editing capabilities. Alu split-gRNA was designed to edit the Rab7a gene as shown in FIG. 4 (with a schematic and initial exemplifications shown in FIGS. 1-3). Experiments were performed to determine the optimum length of the targeting domain. Alu split-gRNAs comprising 50 nt, 100 nt, or 150 nt antisense domains with either Alu75 (split guide A1u75) or Alu225 (split guide Alu225) were designed to edit the Rab7a gene as shown in FIG. 4. The editing capability of split guide A1u75 and split guide Alu225 was compared that of intact guides.

Plasmids were transfected in HEK293 cells. All guides were cloned in a GFP plasmid with no exogenous ADAR added. Approximately 20000 cells were transfected per transfection. 1 microgram of plasmid was used. Cells were harvested for RNA editing analysis 48 hours post transfection.

The first split RNA guides were under the control of a mU6 promoter and the second split RNA guides were under the control of a hU6. The Alu Intact-guides are under the control of a hU6. Left arm guide and right arm guides were transfected alone and then simultaneously. Percent editing was compared to that of the intact guide alone and the 0.100.50 linear guide as a control. Also, a GFP plasmid was used as a transfection control. All guides exhibited RNA editing percentage after cell transfection. Expression of the he first guide RNA in the absence of the second guide RNA can not promote RNA editing due to lack of complementarity with the TAG target site. The second guide RNA had complementary with the TAG site and promoted some editing despite the fact they carried an extensive sequence of Alu element.

As used herein, left arm relates to the first split RNA guide and right arm relates to the second split RNA guide. 50.(75 Alu) refers to the left arm of the guide depicted in the FIG. 50 stands for the number of nucleotides that form perfect complementarity to the target RNA and 75 Alu refers to a 75nt Alu sequence on the 3′ portion of the guide. 100.50.(75 Alu) refers to the right arm of the guide depicted in this figure. The guide is 100 nts in length with an A-C mismatch in the 50th position and has a 75nt Alu sequence at the 5′ portion of the guide. These guides were transfected individually first (the first two data points).

In groups containing both guides, the left and right arms are transfected simultaneously. 50.(Alu75-SPLIT-Alu75)100.50, thus, refers to guide RNAs with a left arm, a right arm, and a split Alu element.

The fourth data point is the intact version of the split guide. 50.(Alu150-Intact). 100.50, thus, refers to to guide RNAs with a left arm, a right arm, and an intact Alu element.

Finally, 0.100.50 is a linear guide with an A-C mismatch at the 50th position. In this experiment, guides were generated that have: variable left arm lengths of 50 nt, 100 nt, 150 nt; SPLIT and INTACT guides with 75 nt Alu sequences (intact 150 nt); and SPLIT and INTACT guides with 225 nt Alu sequences (INTACT 450 nt).

FIG. 4 shows the first guide RNA and second guide RNA form complementary bonds which reconstitutes the ADAR recruiting hairpin for target RNA editing. Intact Alu split gRNA guides performed better when compared to split guide Alu75 in all combinations, while split guide Alu225 performed better when compared to the intact Alu split gRNA guides in all combinations. These results suggest two different promising versions of the Alu split gRNA guide system.

### B. Analysis of Promiscuous RNA Editing

Experiments to analyze promiscuous RNA editing from different combinations are performed.

### C. Reverse Engineering of RNA-guide

Experiments to perform reverse engineering of RNA guide are be performed.

Additional experiments are performed to create a more highly specific Alu-split guided system by introducing bulges to inhibit promiscuous RNA editing.

It is further contemplated that alternative dsRNA (orthogonal sequences) such as Hoppel element mediated dsRNAs (from Drosophila) may be adapted/incorporated into the recombinant guide RNA to increase guide-target specificity.

### D. Analysis of aSHAPE gRNA Editing

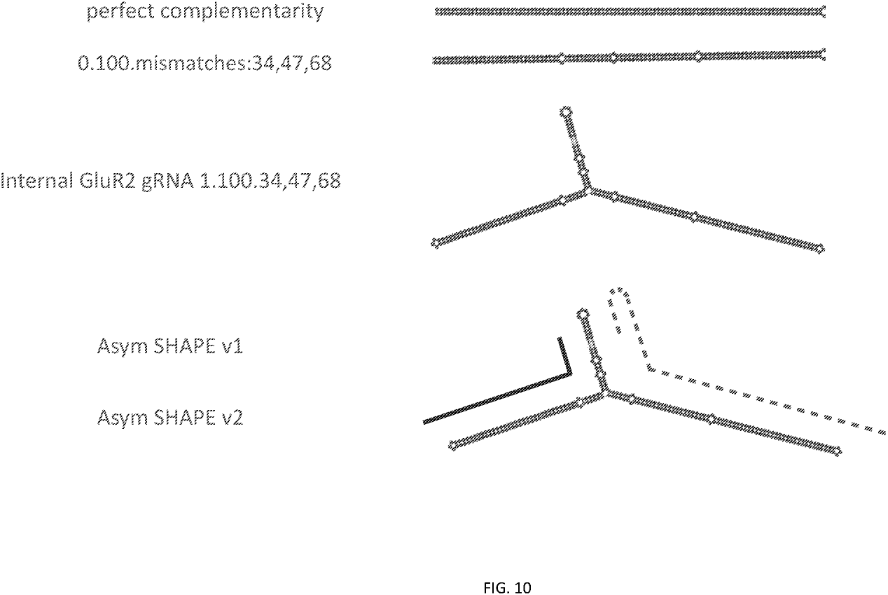

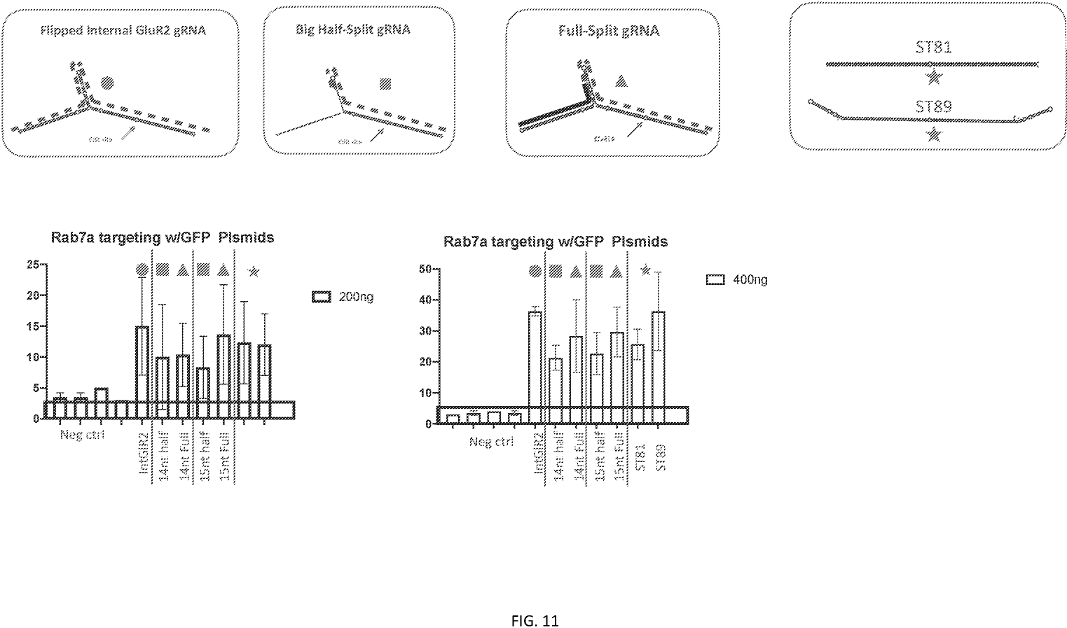

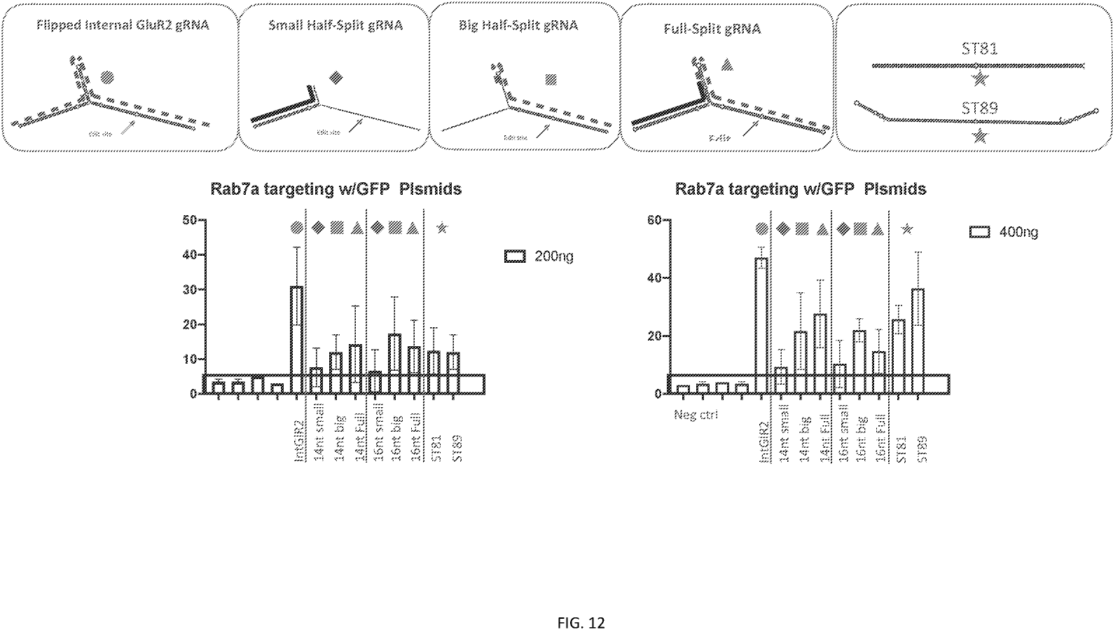

Experiments to determine the capability of aSHAPE gRNA to achieve ADAR editing were performed. FIGS. 6-10 show the various designs used to determine thermostability and activity of test constructs. FIG. 6 demonstrates the higher melting stability of the intact intramolecular GluR2 hairpin compared to asymmetric intermolecular hairpin structures. FIG. 7 demonstrates the trimolecular complex formed between the first guide RNA and second guide RNA and the target RNA. The formation of the trimolecular complex reconstitutes the ADAR recruiting hairpin allowing for ADAR localization to a target RNA, thereby increasing target RNA specificity. FIG. 8 demonstrates the higher thermodynamic stability of the trimolecular complex as compared to the thermodynamic stability of individual guide RNAs and target RNA. FIG. 9 demonstrates that the melting temperature of the intermolecular asymmetric hairpin can be altered by location of the hairpin split location. FIG. 10 demonstrates the various guide designs used to test RNA editing efficiently. FIGS. 11-12 show that aSHAPE gRNA are capable of achieving ADAR editing, especially gRNA1 with 15 nt.

### E. Analysis of GluR2 Hairpin Directionality

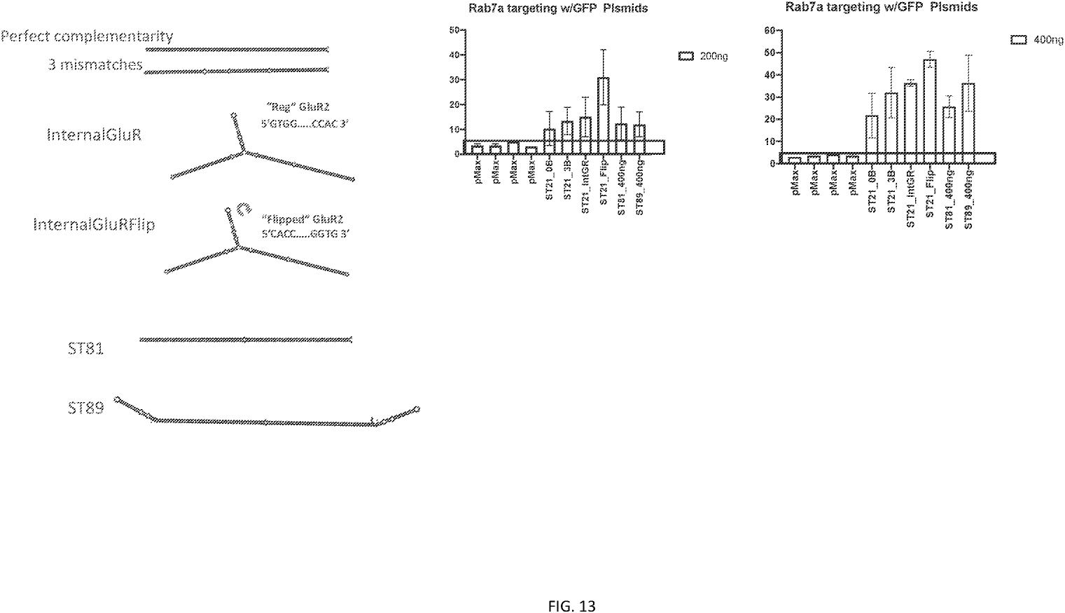

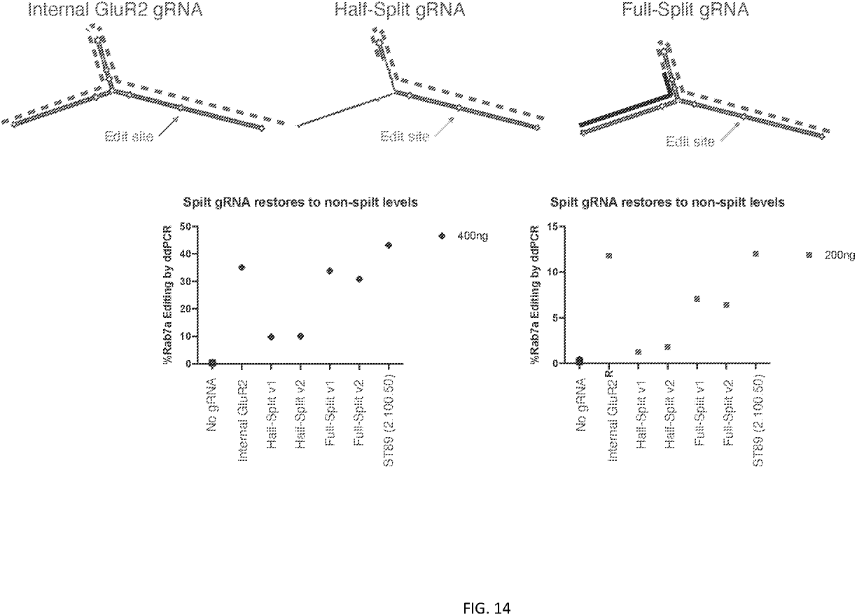

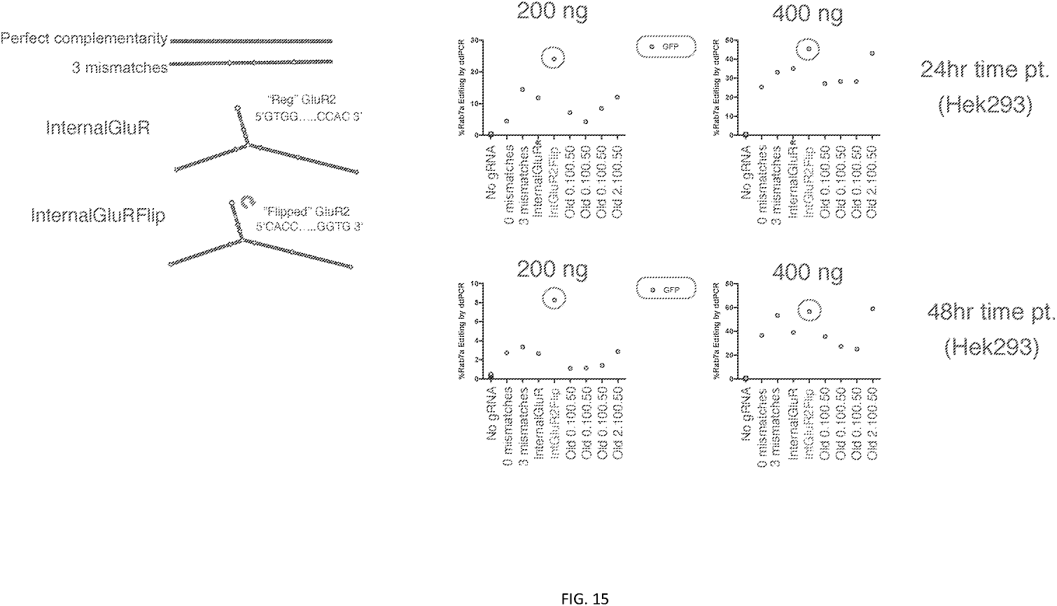

Experiments to determine whether if GluR2 hairpin function is dependent on directionality were performed. FIGS. 13-15 show GluR2 hairpin might have directionality.

### F. Analysis of aSHAPE gRNA Editing Efficiency

Experiments to determine aSHAPE gRNA editing efficiency was performed. aSHAPE gRNA restore to non-split editing level will be compared to internal GluR2.

### 6.10. Sequences

TABLE 1

| SEQ ID NO. | DESCRIPTION | SEQUENCE |
|---|---|---|
| 1 | GluR2 sequence | GUGGAAUAGUAUAACAAUAUGCUAAAUGUUGUUAUAGUAUCCCAC |
| 2 | v1 split, 15 nt fragment | GUGGAAUAGUAUAAC |
| 3 | v1 split, 30 nt fragment | AAUAUGCUAAAUGUUGUUAUAGUAUCCCAC |
| 4 | v2 split, 14 nt fragment | GUGGAAUAGUAUAA |
| 5 | v2 split, 31 nt fragment | CAAUAUGCUAAAUGUUGUUAUAGUAUCCCAC |

### 6.11. Equivalents and Incorporation by Reference

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. §1.57(b)(1), to relate to each and every individual publication, database entry (e.g. Genbank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

What is claimed is:

1. A recombinant adenosine deaminase acting on RNA (ADAR) split guide RNA (adar-sgRNA), comprising:
a first guide RNA comprising a 5′ segment of the ADAR recruiting domain, a 5′ RNA targeting domain capable of binding to a target RNA, or both domains,
a second guide RNA comprising a 3′ segment of the ADAR recruiting domain, a 3′ RNA targeting domain capable of binding to a target RNA, or both domains,
wherein at least one RNA targeting domain has a sequence that is at least partially complementary to the sequence of a segment of the target RNA,
wherein the binding of the first guide RNA and the second guide RNA to the target RNA splints the first and second guide RNAs with the target RNA forming a trimolecular complex, and
wherein the trimolecular complex is capable of recruiting ADAR to deaminate one or more adenosines in the target RNA.

2. The recombinant adar-sgRNA of claim 1, wherein the formation of the trimolecular complex reconstitutes an ADAR recruiting domain comprising the 5′ segment of the ADAR recruiting domain and the 3′ segment of the ADAR recruiting domain.

3. The recombinant adar-sgRNA of any one of claims 1-2, wherein the first guide RNA comprises a first Alu element and the second guide RNA comprises a second Alu element.

4. The recombinant adar-sgRNA of claims 1-3, wherein the first Alu element comprises an oligonucleotide sequence at least partially complementary to an oligonucleotide sequence in the second Alu element.

5. The recombinant adar-sgRNA of claims 1-4, wherein the Alu element comprises an oligonucleotide sequence of from 50-250 nucleotides.

6. The recombinant adar-sgRNA of claim 5, wherein the the Alu element comprises an oligonucleotide sequence of from 50-250 nucleotides.

7. The recombinant adar-sgRNA of claim 5, wherein the Alu element comprises an oligonucleotide sequence of about 225 nucleotides.

8. The recombinant adar-sgRNA of any one of claims 1-2, wherein the ADAR recruiting domain comprises a GluR2 recruiting domain.

9. The recombinant adar-sgRNA of claims 1-8, wherein the ADAR recruiting domain comprises an internal GluR2 loop and/or additional ADAR-recruiting dsRNA loops.

10. The recombinant adar-sgRNA of claim 9, wherein the internal GluR2 loop and/or additional ADAR-recruiting dsRNA loops permit targeting an adenosine in a pre-mRNA or mRNA sequence.

11. The recombinant adar-sgRNA of claim 10, wherein the internal GluR2 loop comprises an asymmetric GluR split gRNA.

12. The recombinant guide adar-sgRNA of any one of claims 1-11, wherein the ADAR recruiting domain has a structure that is capable of binding ADAR1, ADAR2, ADAR3, or any combination thereof.

13. The recombinant guide adar-sgRNA of any one of claims 1-12, wherein the adar-sgRNA comprises a 5′ RNA targeting domain on the first guide RNA.

14. The recombinant guide adar-sgRNA of any one of claims 1-13, wherein the adar-sgRNA comprises a 3′ RNA targeting domain on the second guide RNA.

15. The recombinant guide adar-sgRNA of any one of claims 1-14, wherein the adar-sgRNA comprises both a 5′ RNA targeting domain on the first guide RNA and a 3′ RNA targeting domain on the second guide RNA.

16. The recombinant guide adar-sgRNA of any one of claims 1-15, wherein the at least one RNA targeting domain comprises only 1 mismatched ribonucleotide that is non-complementary to the sequence of the targeted segment of the target RNA.

17. The recombinant guide adar-sgRNA of any one of claims 1-16, wherein the at least one RNA targeting domain comprises a plurality of mismatched ribonucleotides that are non-complementary to the sequence of the targeted segment of the target RNA.

18. The recombinant guide adar-sgRNA of any one of claims 1-17, wherein the at least one RNA targeting domain is from about 20 to about 100 nucleotides in length.

19. The recombinant guide adar-sgRNA of any one of the preceding claims, wherein the first or second guide RNA further comprises a modification at the 5′ and/or 3′ end.

20. The recombinant guide adar-sgRNA of any one of claims 1-19, wherein the target RNA is pre-mRNA, mRNA, non-coding RNA, or viral RNA.

21. The recombinant guide adar-sgRNA of claim 20, wherein the target RNA comprises a mutation that results in altered or modified endogenous protein expression.

22. The recombinant guide adar-sgRNA of claim 21, wherein the target RNA comprises a point mutation, optionally wherein the point mutation results in a missense mutation, splice site alteration, or a premature stop codon.

23. The recombinant guide adar-sgRNA of any one of the preceding claims, wherein the first or second guide RNA has a sequence selected from the group of sequences provided in FIG. 6 and Table 1.

24. A composition comprising at least one recombinant guide adar-sgRNA of any one of the preceding claims, and optionally a pharmaceutically acceptable excipient.

25. A vector, wherein the vector comprises a coding region, wherein the coding region encodes at least one recombinant guide adar-sgRNA encoding the first and second guide RNA of any one of the preceding claims.

26. The vector of claim 25, further comprising expression control elements operably linked to the guide adar-sgRNA coding region.

27. The vector of claim 25 or claim 26, wherein the vector is an AAV or lentiviral vector.

28. The vector of claim 27, wherein the vector is an AAV vector.

29. The vector of claim 28, wherein the AAV vector comprises AAV1, AAV2, AAV3, AAV4, AAV5, AAV6, AAV7, AAV8, AAV9, AAV10, AAV1 1, AAV12, or any variants or chimeras thereof.

30. The vector of claim 28, wherein the guide adar-sgRNA coding region for the first and second guide RNA is operably linked to expression control elements, and the expression control elements and coding region are together flanked by 5′ and 3′ AAV inverted terminal repeats (ITR).

31. The vector of any one of claims 25-30, packaged into a virion.

32. The vector of any one of claims 25-30, formulated in a nanoparticle.

33. A method for modifying the sequence of a target RNA, comprising:
contacting the target RNA with the recombinant guide adar-sgRNA of any one of claims 1 23, the composition of claim 24, or a guide adar-sgRNA expressed from the vector of any one of claims 25-32.

34. The method of claim 33, further comprising delivering at least one polynucleotide, the at least one polynucleotide encoding ADAR1, ADAR2, ADAR3, and any combination thereof.

35. The method of claim 33 or claim 34, wherein the contacting is in vitro or in vivo.

36. The method of claim 35, comprising:
administering the vector of any one of claims 24-30 to a mammalian subject in whom editing of the target RNA is desired.

37. A method of treating a disease or disorder resulting from the decrease or loss of wild-type expression of a protein, comprising:
delivering an effective amount of a recombinant guide adar-sgRNA of any one of claims 1-23, the composition of claim 24, or a guide adar-sgRNA expressed from the vector of any one of claims 25-32, to cells of a patient having a disease or disorder resulting from the loss of wild-type expression of a protein,
wherein the recombinant guide adar-sgRNA is formed by the first and second guide RNA bound to a RNA target in a trimolecular complex which reconstitutes the ADAR recruiting domain, thereby increasing expression of the protein whose expression is decreased or lost.

38. The method of claim 37, wherein the recombinant guide adar-sgRNA is delivered by administering a vector of any one of claims 25-32.

39. The method of claim 37 or claim 38, wherein the target RNA encodes the protein whose expression is altered or modified compared to wildtype expression.

40. The method of any one of claims 37-39, wherein the subject is a human.

41. Use of an effective amount of one or more of the recombinant guide adar-sgRNAs of any one of claims 1-23, the composition of claim 24, or a guide adar-sgRNA expressed from the vector of any one of claims 25-32, for treating a disease or disorder associated with loss of wild-type protein expression.

42. A kit, comprising:
one or more the recombinant guide adar-sgRNAs any one of claims 1-23, the composition of claim 24, or a guide adar-sgRNA expressed from the vector of any one of claims 25-32, and optionally, instructions for use.

43. Any of the recombinant guide adar-sgRNA of claims 1-23, capable of hybridizing to a complementary target ribonucleotide under conditions of high stringency.

44. The recombinant guide adar-sgRNA of claim 43, which hybridizes to a complementary target ribonucleotide under conditions of high stringency equal to or greater than 37° C.


SEQUENCE LISTING (5 sequences)

SEQ ID NO: 1 - RNA, 45 nt, Artificial Sequence
  source: /note="Description of Artificial Sequence: Synthetic oligonucleotide"
guggaauaguauaacaauaugcuaaauguuguuauaguaucccac

SEQ ID NO: 2 - RNA, 15 nt, Artificial Sequence
  source: /note="Description of Artificial Sequence: Synthetic oligonucleotide"
guggaauaguauaac

SEQ ID NO: 3 - RNA, 30 nt, Artificial Sequence
  source: /note="Description of Artificial Sequence: Synthetic oligonucleotide"
aauaugcuaaauguuguuauaguaucccac

SEQ ID NO: 4 - RNA, 14 nt, Artificial Sequence
  source: /note="Description of Artificial Sequence: Synthetic oligonucleotide"
guggaauaguauaa

SEQ ID NO: 5 - RNA, 31 nt, Artificial Sequence
  source: /note="Description of Artificial Sequence: Synthetic oligonucleotide"
caauaugcuaaauguuguuauaguaucccac